(19) United States
(12) Patent Application Publication
(10) Pub. No.: US 2008/0265053 A1
(43) Pub. Date: Oct. 30, 2008

(54) Flying air fresheners

(76) Inventor: Adolfo Guzman, Riverside, CA (US)

Correspondence Address: Gregory Richardson, Suite 210, 3890 11th St., Riverside, CA, 92501, US

(21) Appl. No.: 12/012,516

(22) Filed: Feb. 2, 2008

Related U.S. Application Data
(60) Provisional application No. 60/900,075, filed on Feb. 2, 2007.

(51) Int. Cl. A61L 9/12

(52) U.S. Cl. 239/34; 29/428; 239/211

(57) ABSTRACT

An air freshener is constructed from parts to make a model of an object from everyday life, e.g. an airplane, insect, or bird. The air freshener is made out of a light material such as cardboard, paper, wood, plastic or foam. Other types of models include a space ship, flying dinosaur or other flying object. The scent is sprayed, impregnated, or infused into the body of the air freshener.

This application claims priority from the provisional application No. 60/900,075 filed on Feb. 2, 2007.

### BACKGROUND OF THE INVENTION

1. Field of the Invention

Regular air fresheners are boring and dull to look at, although the scent entices many users. The appearance of most air fresheners deserves hiding, rather than observation. My flying air fresheners are specially designed for viewing while adding a pleasant fragrance to the air. The present invention relates to an air freshener generally and, more particularly, an air freshener in the form of a model of an object of everyday life or a toy. The model functions as a dissipater of a fragrance or scent.

My flying air fresheners were created to offer users a fun way to freshen an area, e.g. the inside of a vehicle. My invention can be placed anywhere and will appear to fly, either by hanging freely or banking, swaying when placed in front of a stream of air, e.g. in air currents circulating in any area.

2. Description of the Related Art

Various air fresheners have been disclosed and are currently on the market and being sold. These air fresheners may be fastened to a flat surface inside a vehicle, such as the dashboard. Other air fresheners may be attached in front of an output port of the air conditioner or heater. In either case, the spread of the scent is limited by the stationary position of the air freshener itself or its inefficient interaction with the air current. Moreover, due to the static nature of these air fresheners, the consumer's attention is not easily obtained.

### SUMMARY OF THE INVENTION

The present invention has been accomplished under the circumstances in view in the accompanying figures.

It is one object of the present invention to provide an air freshener that attracts the attention of a vehicle's occupants as well as people within a room. This is accomplished in one best mode through a model airplane design.

It is another object of my invention to increase the amount of scent or fragrance emitted inside a room or within a vehicle. This is accomplished by increasing the interaction of the model with the flow of the air circulating in an area, e.g. a car or room. The movement of the model airplane increases the amount of air circulating over and around the outside of the model, thus increasing the amount of scent or fragrance emitted into the interior of the vehicle or room.

To achieve these and other objects of my invention the air freshener consists of a model that is assembled by the user by putting together parts. For example, a model airplane air freshener consists of a fuselage part and a wing part. After assembly by the user, the model airplane can hang freely from an object so it appears to be in flight. It also may appear to be in motion when the air circulating in an area moves the plane. The scent or fragrance is impregnated into or sprayed on the surfaces of the model parts, as is known in the art.

My invention contemplates versions in the form of airplanes, jet planes, birds, insects, space ships, kites, and even flying dinosaurs.

### BRIEF DESCRIPTION OF THE DRAWINGS

FIG. 1 is view of an air freshener in the form of a model airplane with attaching device.

FIG. 2 demonstrates the parts that are put together to make a model airplane air freshener.

FIG. 3 shows the parts of the model airplane being put together.

FIG. 4 shows the model airplane freshener attached to a mirror inside of a vehicle.

FIG. 5 shows an alternative form of the invention as a model bird.

FIG. 6 shows an alternative form of my invention as a butterfly or insect.

FIG. 7 shows the parts of a model airplane perforated within the construction material.

FIG. 8 shows various slot configurations.

### DETAILED DESCRIPTION OF THE PREFERRED EMBODIMENT

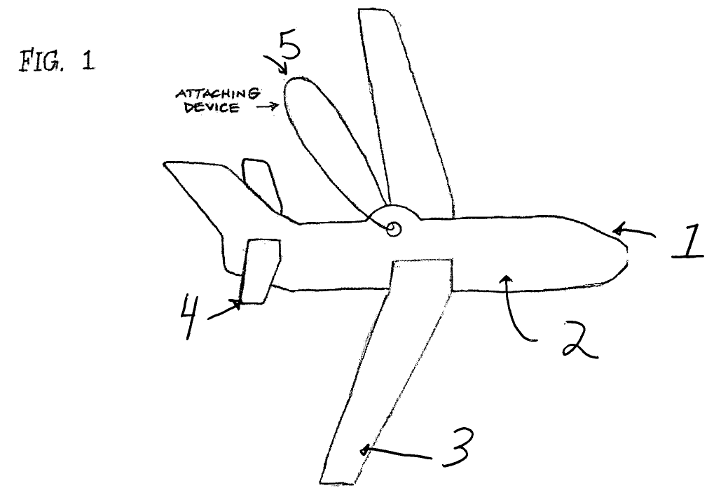

Referring to FIG. 1, an air freshener in accordance with my present invention is shown comprising a model airplane (1) with a fuselage part (2), a wing part (3), and a horizontal stabilizer (4). The attachment device (5) suspends or mounts the model.

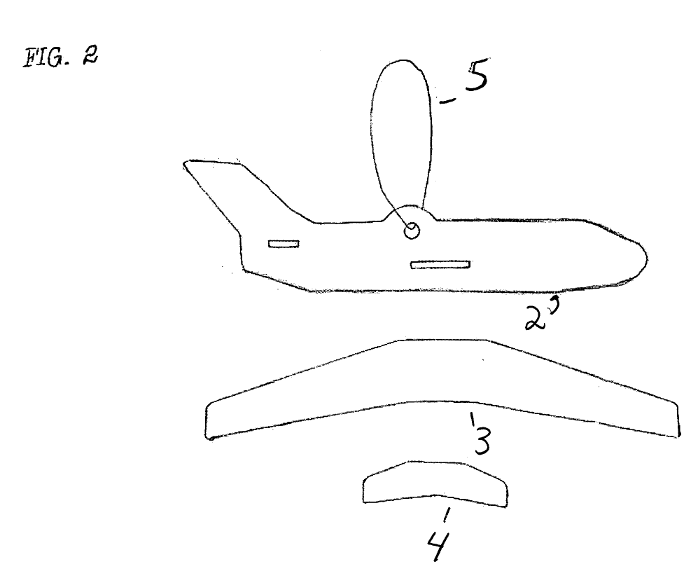

Referring to FIG. 2, showing individual parts of a model airplane air freshener, i.e. the fuselage (2), the wing (3), and the horizontal stabilizer (4) along with the attachment device (5).

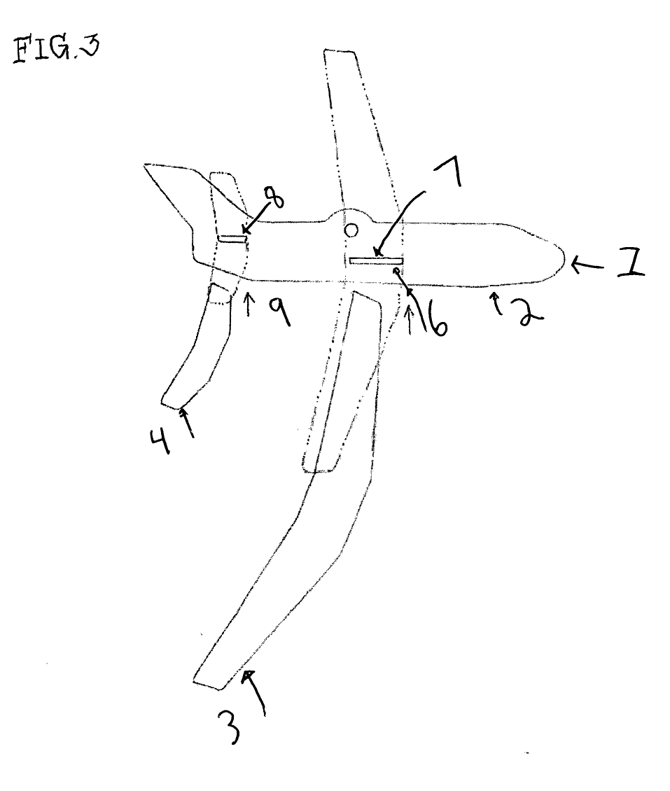

Referring to FIG. 3 a model airplane (1) air freshener is put together by placing the wing part (3) through the fuselage (2), as shown (6). The wings (3) are attached to the fuselage through a wing slot (7) in the fuselage (2). The horizontal stabilizer (4) is attached to the fuselage through another slot (8), as shown (9).

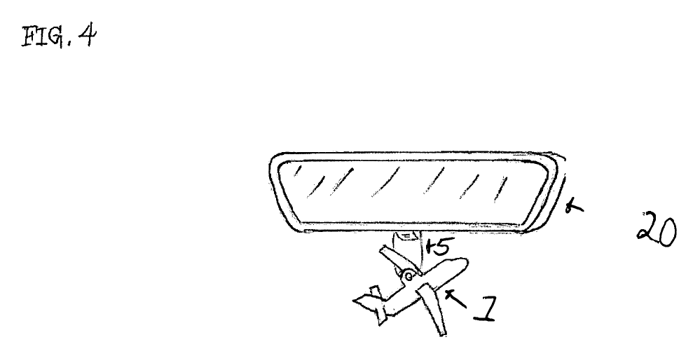

Referring to FIG. 4, the model air plane (1) air freshener is attached by an attachment device (5) to the a mirror (20), in which mode the model airplane swings freely, thereby attracting the interest of the occupants and increasing the flow of air over and around the surfaces of the air freshener.

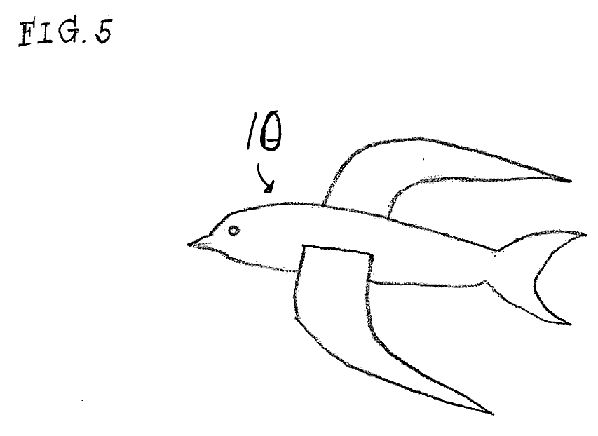

Referring to FIG. 5, the model bird (10) air freshener.

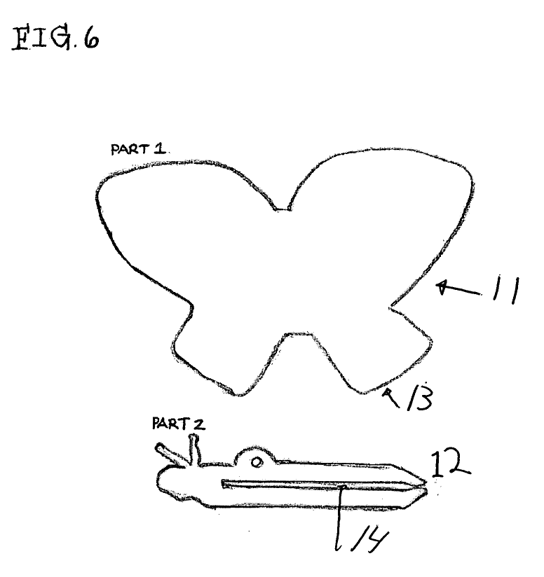

Referring to FIG. 6, the model butterfly (11) or insect air freshener is put together by inserting a wing part (13) through a slot (14) in the body (12).

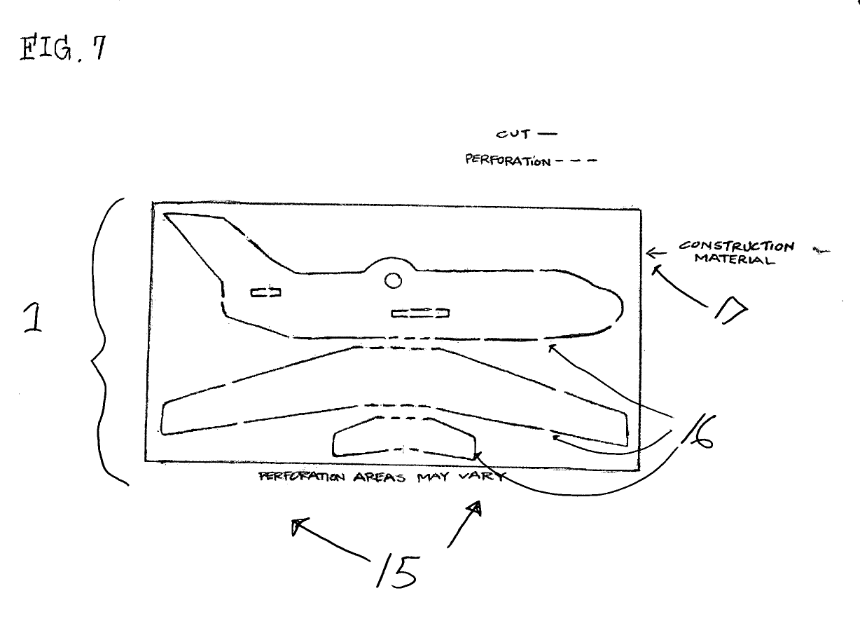

Referring to FIG. 7, the parts to a model airplane (1) air freshener are shown in a kit (15) wherein the parts to the airplane are perforated (16) in the construction material (17).

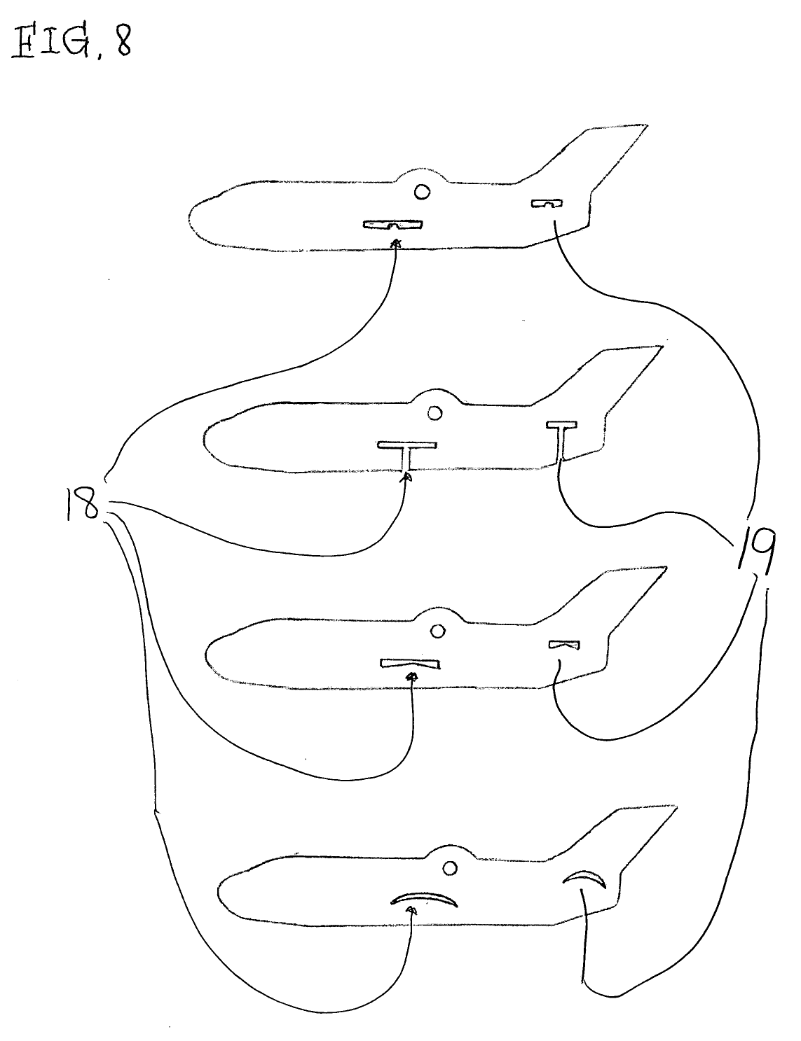

Referring to FIG. 8, various types of slots are shown that accommodate different shapes of wings (18) and horizontal stabilizers (19).

The construction material of my air freshener is preferably made of air freshener cardboard paper, wood, cardboard, plastic, foam or paper. In a preferred embodiment, the parts of the model are perforated, but not cut out entirely, but can be separate pieces. In another preferred embodiment, the construction material is balsa wood. However, other light construction materials are contemplated.

As indicated above, the user can attach the model airplane air freshener by hanging the invention from a mirror or other mount inside the vehicle or room. Then the scent is dissipated throughout the interior as normally happens. When the model airplane air freshener is attached to the rear-view mirror, the oscillations and swaying of the model airplane increases the dissipation of scent by increasing the airflow over the surfaces of the model airplane.

In its best mode, the invention is suspended to that it freely swings through the air so that even more air flows over the surfaces of the air freshener. In each of these embodiments and uses, the interest of the user grows, especially when the mode airplane air freshener appears to bank, sway, and fly on air currents circulating through an area.

A prototype of my invention in the mode of a model airplane air freshener has been constructed with the features of FIGS. 1 through 8. The air freshener functions smoothly to provide all of the features discussed earlier. Although particular embodiments of the present invention have been described in detail for purposes of illustration, other various embodiments using the same principles described herein are contemplated. Additional modifications and enhancements may be made without departing from the spirit and scope of my invention, e.g. by making an air freshener with the appearance of a bird, insect, space ship, jet plane, kite, flying dinosaur, or other animal or object.

What the invention claimed is:

1. An air freshener kit comprising a model object made from at least one part, said parts made from light construction material.

2. The kit of claim 1, wherein the parts are stamped, cut by a laser, or perforated.

3. A kit of claim 2, further comprising an attachment device.

4. A kit of claim 3, wherein said model object comprises at least two of said parts.

5. A method of making an air freshener using a kit of claim 4, comprising the steps of making a model object from the at least two parts.

6. A method of mounting an air freshener of claim 5, comprising the step of securing the air freshener by securing the attachment device to a mount.

7. A method of using an air freshener of claim 6, wherein the attachment device is at least one string-like component.

8. A method of mounting an air freshener of claim 7, comprising the step of suspending the air freshener by the at least one string-like component.

9. A method of using an air freshener of claim 8 to spread scent in an area, comprising the step of claim 8.

10. A method of spreading scent in a room, comprising the method of claim 8, wherein the area is the room.

11. A method of spreading scent comprising the method of claim 8, wherein the area is the inside of a vehicle.

12. A method of using an air freshener of claim 11, wherein the mount is a rear-view mirror.

13. A method of using an air freshener of claim 9 to spread scent in an area, wherein the area is the inside of a room.

14. A method of using an air freshener of claim 8, wherein the mount is located inside of a room.

15. An air freshener of claim 4, wherein said model object is a model airplane and at least one part comprises a fuselage part and a wing part.

16. An air freshener of claim 4, wherein said model object is a model airplane and at least one part comprises a fuselage part, a wing part, and a horizontal stabilizer.

17. A method for making an air freshener of claim 15, comprising the steps of forming a model airplane by placing the wing part through an opening in the fuselage part to form an object with the general appearance of an airplane.

18. A method for making an air freshener of claim 16, comprising the steps of forming a model airplane by placing the wing part and horizontal stabilizer part through openings in the fuselage to form an object with the general appearance of an airplane.

19. A method for making an air freshener of claim 4, wherein said model object is a bird and the at least two parts comprise a body part and a wing part.

20. A method for making an air freshener of claim 4, wherein said model is an insect and the at least two parts form an object with the appearance of an insect.

21. A method for making an air freshener of claim 20, wherein the insect is a butterfly.

22. A means for providing a three (3) dimensional air freshener, comprising the air freshener of claim 1.

23. An air freshener of claim 1, wherein said parts are made of wood, paper, cardboard, foam, or plastic.
